(19) United States
(12) Patent Application Publication
(10) Pub. No.: US 2011/0111094 A1
(43) Pub. Date: May 12, 2011

(54) PROCESS FOR THE PREPARATION OF VEGETABLE PRESERVES CONTAINING PROBIOTIC MICROORGANISMS

(75) Inventors: Paola Lavermicocca, Bari (IT); Stella Lisa Lonigro, Bari (IT); Francesca Valerio, Bari (IT); Angelo Visconti, Bari (IT); Sebastiano Vanadia, Bari (IT); Nicola Calabrese, Bari (IT)

(73) Assignee: Consiglio Nazionale Delle Ricerche, Roma (IT)

(21) Appl. No.: 11/663,965

(22) PCT Filed: Sep. 27, 2005
(86) PCT No.: PCT/EP2005/010413

§ 371 Date: Aug. 12, 2008

(30) Foreign Application Priority Data
Oct. 5, 2004 (IT) MI2004 A 001887

(51) Int. Cl. A23L 1/212; C12N 1/20

(52) U.S. Cl. 426/61; 435/252.9

(57) ABSTRACT

The present invention relates to a process for the preparation of vegetable preserves, comprising immersing a vegetable in a solution containing darkening inhibitors, half cooking the vegetable in water at a temperate of between 90 and 100° C., immersing the vegetable in a brine solution containing probiotic lactobacilli and/or bifidobacteria in a sterile container and sealing the container. The invention further relates to vegetable and vegetable preserves obtained by means of the process.

### FIELD OF THE INVENTION

The present invention relates to food preserves, in particular to vegetable preserves.

### BACKGROUND OF THE INVENTION

Probiotic foods are in general fermented foods containing living and active microorganisms in amounts sufficient to reach the intestine and re-equilibrate the intestinal microflora. Probiotic intake stimulates the growth of beneficial microorganisms, reduces the amount of pathogens and reinforces the body's defenses. It is indeed acknowledged that probiotics, in particular lactobacilli and bifidobacteria, contribute to maintain the equilibrium of the intestinal flora (Salminen S., et al. Int. Dairy J. 8:563, 1998; Saarela M., L. et al., Int. J. Food Microbiol. 2002, 78:99), are able to inhibit pathogens (Drago L., M. R. et al., FEMS Microbiol. Letters, 1997, 153:455 e Cross M. L. FEMS Immunol. Med. Microbiol. 2002, 34:245) and therefore protect the body from gastro-intestinal diseases. When the intestinal microflora is altered, non only the administration of probiotics re-establishes the normal equilibrium, but it also improves the microbial balance and the properties of the endogenous microflora. Moreover, there are experimental evidences on the role of probiotics in the prevention of food allergies and intolerances (Isolauri E., et al., Am. J. Clin. Nutr. 2001, 73 (suppl.): 444s-; Jahreis G., et al. Food Res. Int. 2002, 35:133).

As far as products for human nutrition are concerned, probiotic bacteria are primarily added to fermented milk, especially yogurt. One of the problems of the preparation of probiotic foods is the influence of the production technologies on the properties of the concerned strains, in particular cell viability and integrity, as well as population dimension and stability (Mattila-Sandholm T., et al. Int. Dairy, 2002 J. 12: 173). Liquid and frozen cultures have been widespreadly used in the past, but their production, transport and storage are highly expensive. Lyophilized cultures are widely used at present, but the cells are often damaged and cannot be stored for long periods. In fact, lyophilized cells survive in anabiosis and viability is restored by rehydration; however, rehydration does not guarantee complete cell survival, and can also alter cell metabolism or make cells unable to stand gastric acidity. Concentrated cell cultures in monodose vials are also very common and they can be found on the market either fresh or frozen. A major difficulty in this case is bacterial multiplication, which is necessary to reach high concentrations, i.e. about 10¹⁰ colony forming units (CFU)/g. Therefore, most probiotic products are nowadays of animal origin, in particular dairy products such as yogurt, cheese, desserts and ice-creams. In some cases dairy products, especially yogurt, are added with alimentary fibers, in particular insoluble fibers, for example inulin, which exert a pre-biotic function, i.e. selectively stimulate the growth of probiotic bacteria in the colon. However, the intake of dairy products may be problematic for some people, due to allergies and/or intolerances to milk and its-derivatives.

Dehydrated fruits impregnated under vacuum with probiotic microrganisms have been obtained on an experimental scale (Betoret N., et al. J. Food Engin. 2003, 56: 273), while oat-based products and fruit juices containing probiotic microorganisms (Johansson et al. Int. J. Food Microbiol., 1998, 42:29) are already on the market; all these products must be rapidly consumed after opening.

It would therefore be advantageous to provide vegetable foods for the administration of probiotic bacteria, which can be stored for long periods without significant alteration of their organoleptic and nutritional properties. Usually plant products, in particular vegetables, can also be preserved i) in brine ii) in vinegar or (iii) in oil. Fresh or semi-processed vegetables can be used.

For the preparation of preserves in brine, the edible portion of the vegetable is properly rinsed, soaked in brine and placed in sterilizable containers, which are immediately hermetically sealed and sterilized according to procedures and times that depend on the product and the size of the container. Usually, sterilization is carried out with boiling water or steam, for a time ranging from 15 to 60 minutes. This preserving method often determines overcooking, which is detrimental both to organoleptic properties (aspect, consistency, taste) and nutritional values. For preservation in vinegar, fresh or semi-processed vegetables, i.e. soaked in a highly saline solution (8-10% NaCl, sometimes even up to 25%) until canning, are processed and put in vinegar. Sometimes, to improve organoleptic properties and preservation, additives may also be added. All these steps determine radical changes of the organoleptic properties, especially taste and smell, and also a decrease in nutritional substances. For preservation in oil, the concerned vegetable, either acidified, cooked or half-cooked, is placed in suitable containers and added with oil; the vegetable must be well-soaked in oil, in order to create an oxygen-free environment and prevent bacterial proliferation. The finished product is sterilized or pasteurized to ensure preservation. However, also preservation in oil causes a decrease of nutritional substances. Moreover, the organoleptic properties of in-oil products are very different from the fresh ones and even if drained of oil they are highly caloric.

### DETAILED DESCRIPTION OF THE INVENTION

A process that allows to prepare vegetable preserves with a high content of probiotic microorganisms has now been found. The process comprises addition of probiotic microorganisms to plant products, in particular vegetables, it does not require sterilisation or preservation in vinegar or oil and provides preserves which can be stored at room temperature (about 25° C.) for a relatively long-term period.

Accordingly, the present invention relates to a process for the preparation of vegetable preserves containing probiotic microorganisms comprising immersing a vegetable, suitably sized and without non-edible parts, in an aqueous solution containing substances that prevent darkening, preferably citric acid, ascorbic acid, lemon juice or vinegar; draining and half-cooking the vegetable in water at a temperature ranging from 90 to 100° C.; soaking the vegetable in a brine solution containing lactobacilli and/or bifidobacteria in a sterile container and sealing the container.

The concentration of lactobacilli and/or bifidobacteria is higher than 10⁶ and is preferably comprised between 10⁸ and 10⁹ CFU/ml. The lactobacilli are preferably selected from Lactobacillus paracasei and Lactobacillus plantarum, more preferably Lactobacillus paracasei IMPC2.1 (deposited with the Belgian Coordinated Collections of Microorganisms, BCCM/LMG-Collection, Gent, Belgium, accession number LMG P-22043) and Lactobacillus plantarum ITM21B (deposited with the Belgian Coordinated Collections of Microorganisms, BCCM/LMG-Collection, Gent, Belgium, accession number LMG P-22033) are used. Lactobacillus paracasei LMG P-22043 was disclosed for the first time in patent application MI2003A002391, while Lactobacillus plantarum LMG P-22033 is new and is a further object of the invention.

According to a preferred embodiment of the invention, the concentration of NaCl in the brine is comprised between 4 and 5%; according to a more preferred embodiment, the concentration is 4%.

Further to probiotic microorganisms, the brine solution can also contain spices and/or aromas.

The concentration of darkening-preventing substances, for instance citric acid or ascorbic acid, is comprised between 0.5 and 1.5% and is preferably of 1%.

Even though any vegetables are suitable for carrying out the present invention, chicory, carrots, beetroot, potatoes, cauliflowers, wild onions (lampagioni), Bruxelles sprouts, onions, tomatoes, peppers, aubergines, zucchini, cucumber, capers, mushrooms, asparaguses and artichokes are preferred. According to a particularly preferred embodiment of the invention artichokes are used.

Accordingly, the invention relates to vegetable preserves obtainable with the process described above. Artichokes preserves are preferred, in particular those containing probiotic lactobacilli, such as Lactobacillus plantarum and Lactobacillus paracasei, more particularly Lactobacillus plantarum LMG P-22033 and Lactobacillus paracasei LMG P-22043.

The artichokes preserved according to the process of the invention contain, on the brats and receptacle, a number of microorganisms higher than 10⁶ (comprised between 3.6×10⁶ and 6.2×10⁷ CFU per gram after 3-months storage), i.e. higher than 10⁸ CFU (1.4×10⁸ and 2.4×10⁹ CFU) per head weighing about 40 g (Tables 1 and 2).

For the preparation of artichokes preserves, artichokes head are sized, rounded off and after cutting off the apex and the brats, soaked in a citric acid solution (1%). After half-cooking (about 5 minutes) in water at 90-100° C., they are manually placed in pots under sterile conditions and soaked in a low-concentration brine (4% NaCl upon packaging) containing a suspension of probiotic bacteria, so as to obtain a final concentration higher than 10⁶ CFU per ml of brine. The product is not submitted to further treatments.

The process of the invention ensures high organoleptic and nutritional properties of the products since, due to the mild thermal treatment, the polyphenols, vitamins and fibres content is safeguarded; moreover, probiotic microoroganisms provide additional functional properties. The quality of the vegetables preserved in this way (consistency, absence of darkening, natural color etc.) is very similar to that of seasonally prepared home-made preserves. The preserves are as pleasant as half-cooked vegetables and contain a proper ratio of acids and simple sugars (mainly fructose, which is released on the vegetables by the bacteria) (FIG. 1); moreover, contrary to products preserved in brine, those of the invention are tasty, but not salted, because the presence of microorganisms allows to use a limited amount of salt.

Preserves containing L. paracasei LMG P-22043 and Lactobacillus plantarum LMG P-22033 are beneficial due to the ability of these strains to develop both under aerobic and anaerobic conditions, adhere in an even and stable manner, resist to gastric juices and bile salts and colonize the human intestine. All these properties characterise the probiotic role of the food in question.

The two above-mentioned strains are particularly advantageous for the preservation of the product, which can be stored sealed at room temperature up to three months; on the contrary, in control samples without the microbial inoculum, the product is irremediably degraded after 3-4 days. It should be noted that, even if a thermal treatment like sterilisation (which would also be more costly in terms of energy) would ensure longer preserving times, the product would be less acceptable from the organoleptic and nutritional point of view.

The process of the invention allows an immediate control of the quality of the preservation state, since the addition of microorganisms causes a decrease in the pH which, from the value of 5.5 of the product soaked in brine, further decreases to 3.5+3.8 within some hours and remains unchanged during preservation.

The product, which can be stored for more than 90 days, is ready to use and does not require further treatments.

It will also be appreciated that the preserves of the invention can either be consumed as such or used for the preparation of probiotic foods, which are a further aspect of the invention. A further advantage is that the intake of only part of the product (vegetables and not brine) provides a dose of probiotic bacteria comparable to that contained for instance in yogurt or concentrated cultures.

Finally, it should be noted that, differently from probiotic foods wherein microorganisms are suspended in a liquid matrix, on the vegetables, in particular artichokes, the bacterial cells are immobilized, which allows effective and safe transport in the gastro-intestinal tract. Moreover, the binding to a solid product with a very complex framework, such as artichokes, allows the microorganisms to resist the attack of gastric juices.

The preserves obtainable with the process of the invention and the foods containing them are particularly suitable for the prophylaxis and treatment of diseases caused by some food contaminants, gastro-intestinal diseases that affect travellers, as coadjuvant in antibiotic therapies and, more generally, in the situations wherein it is necessary to increase the body's immune defenses.

Since the process of the invention does not comprise a drastic thermal treatment, it does not causes remarkable loss of polyphenols and alimentary fibres in the vegetables. In the case of artichokes, after a 3-month storage, the product has a content (more than 1 g/100 grams of product) of alimentary fibre (inulin) comparable to that of the fresh product and a higher content of (0.1 g) of simple sugars (fructose) per 100 grams of drained product.

The preserves of the invention are suitable for diabetic patients, due to the presence of fructose and the nearly total absence of glucose.

The invention will be now illustrated in more detail in the following experimental section.

### DESCRIPTION OF THE FIGURES

FIG. 1 shows the trend of the sugars concentration (glucose, fructose, sucrose) as a function of time and bacteria.

FIG. 2 shows the adhesion of L. paracasei LMG P-22043 (A) and L. plantarum LMG P-22033 (B) on the surface of artichokes in comparison with non-inoculated artichokes (C). SEM observation.

FIG. 3 shows Lactobacillus paracasei LMG P-22043 tested in simulated digestion as cell suspension () or adhering to artichoke surface (▪).

FIG. 4 shows Lactobacillus plantarum LMG P-22033 tested in simulated digestion as cell suspension () or adhering to artichoke surface (▪).

### EXPERIMENTAL SECTION

### 1) Viability of Probiotic Bacteria on Artichokes Heads

The ability of colonizing artichoke heads and the time-persistence has been evaluated for the following strains: Lactobacillus paracasei LMG P-22043 and Lactobacillus plantarum LMG P-22033.

The tests were performed on artichoke heads processed as described above. Strain viability was evaluated using sterile pots with screw caps containing 8 heads/240 ml of sterile brine (4% NaCl, starting pH 5.95).

Procedure. Previously processed heads were soaked in brine, then added with a bacterial suspension to a final concentration comprised between 10⁸ and 10⁹ colony forming units (CFU) per ml of brine for each singly inoculated bacterium. Non-inoculated heads were used as the control. The samples were stored for 3 months at room temperature (about 25° C.) and ¼ head for each sample was taken at 1, 30, 60 and 90 days and the amount of bacteria was evaluated. At each taking the samples were completely drained, added with 20 ml of NaCl (0.85%) and Tween 80 (0.025%) and vigorously stirred for one hour to allow the detachment of the bacteria from the sample surface. The resulting suspension was seeded onto an agarized substrate for the count of lactic bacteria. The results are reported in the following table (Table 1).

TABLE 1
CFU per gram of product (artichoke heads)

| Strain | Day 1 | 30 days | 60 days | 90 days |
|---|---|---|---|---|
| L. paracasei | 1.32 × 10⁹ ± | 1.24 × 10⁹ ± | 3.70 × 10⁷ ± | 3.59 × 10⁶ ± |
| LMG P-22043 | 4.21 × 10⁸ | 6.21 × 10⁸ | 1.89 × 10⁶ | 1.19 × 10⁶ |
| L. plantarum | 9.18 × 10⁸ ± | 1.94 × 10⁸ ± | 8.85 × 10⁶ ± | 6.16 × 10⁷ ± |
| LMG P-22033 | 1.69 × 10⁸ | 6.37 × 10⁷ | 1.23 × 10⁶ | 3.28 × 10⁷ |

All tests were repeated 3 times with two repetitions. The data reported in the table are the average f standard error (n=6) of the tests. Based on these results, the content of probiotic bacteria in a whole head weighing 40 g is, in both strains, higher that 10⁸ CFU during the whole experiment (see Table 2).

TABLE 2
CFU whole head (mean weight of 40 g)

| Strain | Day 1 | 30 days | 60 days | 90 days |
|---|---|---|---|---|
| L. paracasei LMG | 5.1 × 10¹⁰ | 4.8 × 10¹⁰ | 1.4 × 10⁹ | 1.4 × 10⁸ |
| P-22043 | | | | |
| L. plantarum LMG | 3.5 × 10¹⁰ | 7.5 × 10⁹ | 3.4 × 10⁸ | 2.4 × 10⁹ |
| P-22033 | | | | |

Therefore, the intake of probiotic microorganisms through the vegetable preserves of the invention can be compared to that of concentrated microorganisms cultures or bio-yogurt or other milk-based probiotic products.

### 2) Simple Sugars and Inulin in the Course of the Transformation

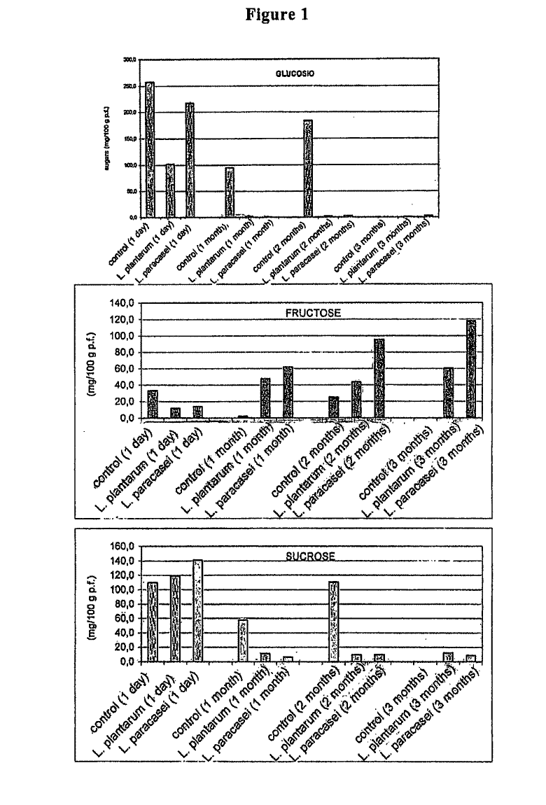

Separate inoculation of lactic bacteria caused a considerable increase in the fructose content with respect to the control, which accounts for the pleasant taste of the treated product. As it can be seen from FIG. 1, such an increase (already evident after 1-month storage) is accompanied by a nearly complete disappearance of glucose and a residual saccharose amount that contributes to make the product sweet.

Moreover, preliminary tests carried out on heads inoculated with L. plantarum showed that the treatment contributes to preserve the starting inulin content, as it involves a mild thermal treatment which ensures a negligible loss of the compound. The high inulin content allows to maintain the prebiotic properties of fresh vegetables.

### 3) Resistance of Probiotic Strains Adhering on Artichoke Surface to Artificial Gastric and Intestinal Fluids

In order to be beneficial for the intestine, the bacteria must survive the passage through the acidic stomach environment (the gastric pH in healthy humans is of about 2-2.5). The resistance to the lytic action of bile salts of the small intestine is another important aspect. Therefore, the potential ability of the strains to survive the passage through the gastrointestinal tract and colonize it can be evaluated by evaluating their resistance to gastric and intestinal juices. However, the survival of each strain could be affected by the protective action of food-carriers. It has been found that some Lactobacilli and Bifidobacteria can tolerate gastric acidity when ingested with milk products (Charteris W. P. et al. 1998. Development and application of an in vitro methodology to determine the transit tolerance of potentially probiotic Lactobacillus and Bifidobacterium species in the upper human gastrointestinal tract. Journal of Applied Microbiology 84: 759). In other studies, a protective action on probiotic populations in the gastro-intestinal tract has been attributed to the matrix and high fat content of cheese (Stanton C. et al. 1998. Probiotic cheese. International Dairy Journal 8:491).

The following results demonstrated that when bacterial cells adhering to artichoke surface were incubated in gastric and intestinal fluids to simulate gastric digestion, their survival ability was improved.

Lactobacillus plantarum LMG P-22033 and Lactobacillus paracasei LMG P-22043 were tested in simulated digestion as cells suspension or adhering to artichoke surface. Briefly, for the former sample, 20 ml of MRS medium were inoculated at 2% (v/v) with both strains and incubated at 37° C. for 24 h. For the latter sample, half-cut strain-bearing artichokes were roughly homogenized. For both samples, after washing in sterile saline solution (0.9% NaCl) and centrifugation, the pellet was added to 20 ml of artificial gastric juice with the following composition (mmol/L): NaCl, 125; KCl 7; NaHCO₃, 45 and pepsin, 3 g/L. The final pH was adjusted to pH 2 with HCl or with to pH 7 NaOH. The bacterial suspensions were incubated with agitation (200 rev min⁻¹) to simulate peristalsis. Aliquots were taken for the count of viable cells at 0 and 180 min. Treatment with intestinal fluids was carried out by suspending the cells (after 180 min of gastric digestion) in 0.1% (wt/v) pancreatin and 0.15% (w/v) Oxgall bile salts in water and adjusting it to pH 8.0. The suspensions were incubated as above and samples for total viable counts were taken at 0 and 180 min. (Method adapted from Fernandez M. F. et al. 2003. Probiotic properties of human lactobacilli strains to be used in the gastrointestinal tract. Journal of Applied Microbiology, 94: 449).

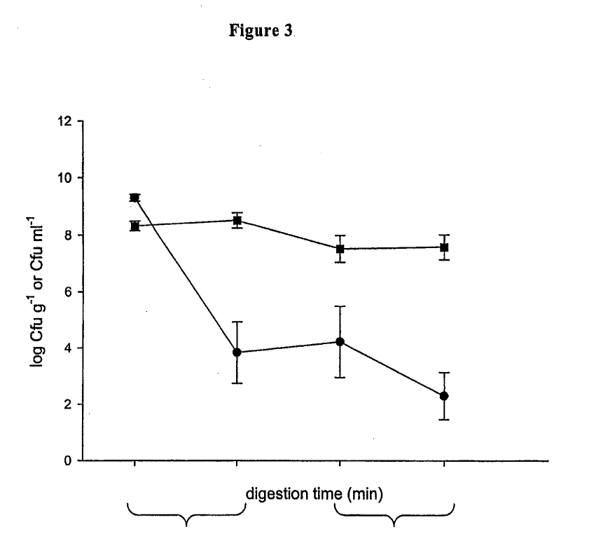

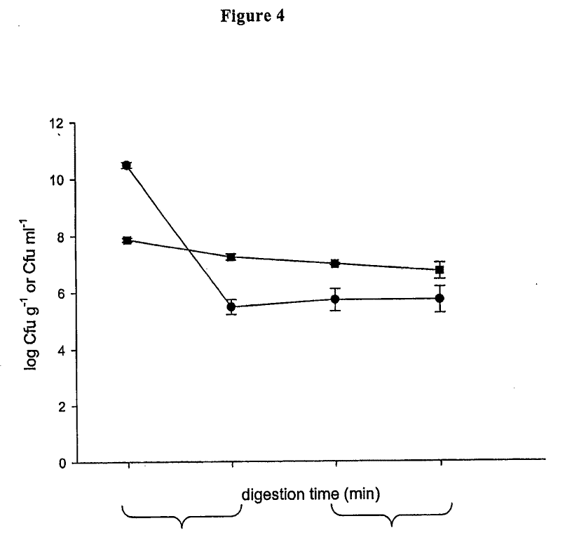

In the absence of adhesion to the vegetable surface, a relevant decrease in viability was observed for both strains after 3 h of incubation in the presence of gastric juice (pH 2). When bacterial cells adhering to artichokes were tested, a complete recovery of the population was obtained after incubation at pH 2. Similar results were obtained with intestinal fluid. The entire population of both strains adhering to the artichoke's surface survived in acidic conditions for 3 h and was not lysed in the presence of bile salts. The results, summarized in FIGS. 3 and 4, demonstrate the protective effect of artichokes and that the survival of probiotic strains can be modulated by their adhesion to this food-carrier.

### 4) Persistence of L. paracasei IMPC 2.1 in the Gastro-Intestinal Tract

Artichokes containing L. paracasei LMG P-22043, prepared as described on page 4 of the description), were administered to volunteers to verify the ability of the artichokes of the invention to deliver the probiotic bacterial cells to the gastrointestinal tract. Four healthy adult subjects were fed for 10 days with portions of 2 artichoke heads, thoroughly drained, containing in all 2×10¹⁰ UFC of L. paracasei IMPC 2.1. The composition of the intestinal flora of the subjects was monitored at the beginning (time 0), after 10 days (t=10) of administration and after 7 days from the end of administration. At each sampling, 1 gram of faeces from each subject was added with 9 ml of Amies medium, homogenized and submitted to decimal dilutions, then plated on a 12 μg/ml Rogosa±vancomycin substrate and cultured in anaerobiosis for 48 hours at 37° C.

Ten-twenty percent of total colonies randomly selected from countable SL-Rogosa-vancomycin agar plates were isolated, checked for purity and DNA was extracted. A Repetitive Extragenic Palindromic-PCR (REP-PCR) analysis was performed (primer: (GTG)₅ (5′-GTG GTG GTG GTG GTG-3′) according to the method of Versalovic et al (1994, Methods in Molecular and Cellular Biology, 5:25-40).

TABLE 3
Lactic populations in human subjects before and after
administration of artichokes added with L. paracasei LMG P-22043.

| | Lactobacillus spp. | L. paracasei |
|---|---|---|
| | vancomycin resistant | LMG P-22043 |
| | (cfu/gr faeces) | (cfu/gr faeces) |
| t = 0 | | |
| Subject 1 | 1.0 × 10⁵ | 0 |
| Subject 2 | 8.9 × 10⁵ | 0 |
| Subject 3 | 2.7 × 10⁷ | 0 |
| Subject 4 | 1.2 × 10⁶ | 0 |
| t = 10 | | |
| Subject 1 | 1.7 × 10⁷ | 1.1 × 10⁷ |
| Subject 2 | 1.2 × 10⁷ | 2.5 × 10⁵ |
| Subject 3 | 1.0 × 10¹⁰ | 1.0 × 10¹⁰ |
| Subject 4 | 5.1 × 10⁷ | 5.1 × 10⁷ |
| F.O. | | |
| Subject 1 | 1.5 × 10⁶ | 0 |
| Subject 2 | 1.5 × 10⁷ | 0 |
| Subject 3 | 2.6 × 10⁷ | 0 |
| Subject 4 | 1.5 × 10⁴ | 0 |

An increase of about two logarithmic cycles in the intestinal lactic population of the four subjects was observed. The isolated colonies were subjected to molecular identification whereby it was ascertained that L. paracasei IMPC 2.1 was present in all subjects and colonized the intestine.

### 5) Adhesion to Artichoke Surface

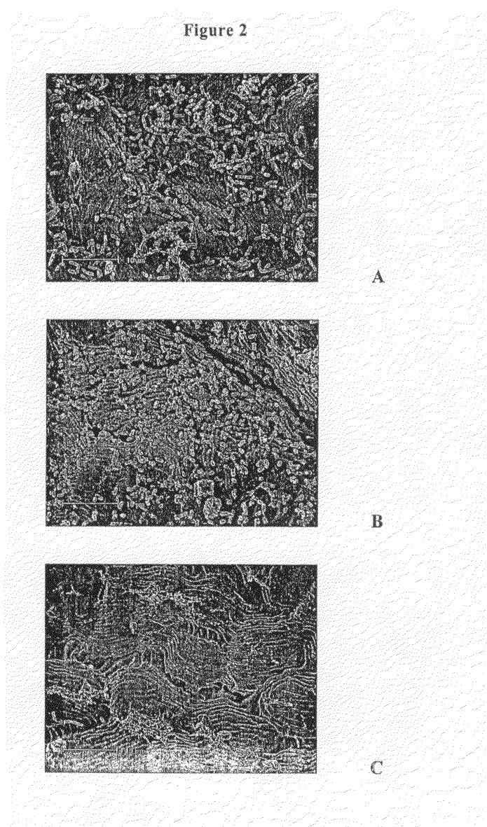

Scanning electron microscopy (SEM) observations were performed to assess the ability of the strains used in this study to adhere to artichoke surface. The results are reported in FIG. 2.

### 6) Viability of Probiotic Strains on Different Artichoke Varieties

Colonization and survival of L. paracasei LMG P-22043 and L. plantarum LMG P-22033 on the surface of two different artichoke varieties (Opal and Concerto) was evaluated. Tests were carried out on fresh artichokes processed as described on page 4 of the description. The results are reported in the following table:

TABLE 4
UFC per gram of artichoke head ± standard error

| Artichoke | | | | |
|---|---|---|---|---|
| variety | Strain | 1 day | 30 days | 60 days |
| Opal | L. paracasei | 2.6 × 10¹⁰ ± | 2.0 × 10⁹ ± | 1.0 × 10⁸ ± |
| | LMG P-22043 | 1.3 × 10⁹ | 9.0 × 10⁸ | 8.8 × 10⁶ |
| | L. plantarum | 2.3 × 10⁹ ± | 1.0 × 10⁸ ± | 1.2 × 10⁷ ± |
| | LMG P-22033 | 1.7 × 10⁸ | 6.4 × 10⁷ | 5.8 × 10⁵ |
| Concerto | L. paracasei | 5.7 × 10⁸ ± | 4.4 × 10⁸ ± | 3.6 × 10⁷ ± |
| | LMG P-22043 | 4.1 × 10⁸ | 6.3 × 10⁷ | 1.4 × 10⁶ |
| | L. plantarum | 4.5 × 10⁸ ± | 9.2 × 10⁷ ± | 1.8 × 10⁷ ± |
| | LMG P-22033 | 1.8 × 10⁸ | 3.0 × 10⁷ | 6.8 × 10⁶ |

The strains showed a high survival rate for 60 days on both artichokes varieties, the higher being on the Opal variety.

### 7) Colonization and Survival of Lactobacilli and Bifidobacteria on Semi-Finished and Commercially Available Ready-To-Eat Vegetables

Generally vegetables are stored as semi-finished pasteurized products in brine until marketing. Tests were carried out to evaluate the ability of different semi-finished (artichokes, zucchini) or ready to eat (asparagus, mushrooms) brined vegetables to be colonized by lactobacilli (L. paracasei LMG P-22043, L. plantarum LMG P-22033) and bifidobacteria (Bifibacterim bifidum ATCC15696) and avoid the final sterilization step.

### Vegetable Samples

Semi-finished artichokes: whole artichokes pasteurized and preserved in brine (about 5% NaCl, pH 4.2). Artichoke heads (about 320 gr) were placed in 500 ml sterile glass jars and covered with their own brine diluted with sterile water to obtain a final concentration of 4% NaCl.

Semi-finished zucchini: sliced zucchini, slightly cooked and preserved in brine (about 2.5% NaCl, pH 3.8) containing lactic, citric and ascorbic acid. Sliced zucchini (about 350 g) were placed in 500 ml sterile glass jars and covered with their own brine.

Ready-to-eat asparaguses: green asparaguses preserved in brine (about 1% NaCl, pH 5.31) containing citric acid. Asparaguses (about 380 g) were placed in 500 ml sterile glass jars and covered with their own brine.

Ready-to-eat mushrooms: cultivated mushrooms pieces and stems preserved in brine (about 1% NaCl, pH 4.90) containing citric and ascorbic acid. Mushrooms (about 380 gr) were placed in 500 ml sterile glass jars and covered with their own brine.

All vegetables were added with a bacterial suspension containing about 10⁹-10¹⁰ CFU ml⁻¹. At the inoculation time and after 30, 60 and 90 days of incubation at room temperature, bacterial counts were performed as previously described. Strains survived on all vegetables as shown in the following table:

TABLE 5
UFC per gram of vegetables

| | | | | | Under |
|---|---|---|---|---|---|
| | Strain | 1 day | 30 days | 60 days | course |
| Semi- | L. paracasei | 1.2 × 10⁸ | 7.4 × 10⁸ | 2.9 × 10⁸ | |
| finished | LMG P-22043 | | | | |
| arti- | | | | | |
| chokes | | | | | |
| Semi- | L. paracasei | 9.7 × 10⁷ | 2.8 × 10⁸ | 1.7 × 10⁷ | |
| finished | LMG P-22043 | | | | |
| zucchini | | | | | |
| | | | | | 90 days |
| Ready- | L. paracasei | 8.0 × 10⁷ | 6.4 × 10⁷ | 1.0 × 10⁷ | 1.3 × 10⁵ |
| to-eat | LMG P-22043 | | | | |
| aspara- | L. plantarum | 1.2 × 10⁸ | 1.0 × 10⁷ | 4.6 × 10⁴ | 0 |
| guses | LMG P-22033 | | | | |
| Ready- | L. plantarum | 2.4 × 10⁸ | 1.2 × 10⁷ | 1.7 × 10⁵ | 2.1 × 10⁵ |
| to-eat | LMG P-22033 | | | | |
| mush- | B. bifidum | 3.4 × 10⁵ | 6.6 × 10⁶ | 4.1 × 10⁵ | 4.4 × 10⁴ |
| rooms | ATCC15696 | | | | |

It was observed that L. paracasei strains had a high survival rate during the experimental period on semi-finished artichokes with a population of about 10⁸ UFC per gram (similar to that observed on the freshly prepared artichokes of the present invention). Also on semi finished zucchini, a high survival rate during the experimental period was observed with populations of 10⁸ and 10⁷ UFC per gram after 30 and 60 days respectively. The strain L. paracasei greatly survived also on asparaguses with populations ranging from 10⁷ to 10⁵ UFC per gram.

The L. plantarum strain showed a good survival rate both on asparagus and mushrooms until 60 days.

The survival observed for B. bifidum indicated that the strain survived on mushrooms with populations fluctuating around 10⁵ UFC per gram. This result is indicative of a successful introduction of bifidobacteria in the vegetable food chain.

### 8) Organoleptic Properties

The artichoke preserve was evaluated by tasters and resulted pleasant as far as taste was concerned, the natural color was maintained and no darkening was observed. The artichokes preserved according to the invention are as pleasant as half-cooked vegetables. Moreover, contrary to industrially transformed products preserved in brine (i.e. without vinegar or oil added), they are tasty, but not salted, since the microorganisms allow to employ a reduced amount of salt.

To summarize, the products show harmonized properties that cannot be found in the corresponding home-made, cottage or industrial products.

1. A process for the preparation of vegetable preserves, comprising immersing a vegetable in a solution containing darkening inhibitors; half-cooking the vegetable in water at a temperature between 90 and 100° C.; immersing the vegetable in a brine solution containing probiotic lactobacilli and/or bifidobacteria in a sterile container and sealing the container.

2. The process according to claim 1 wherein the probiotic lactobacilli and/or bifidobacteria concentration in the brine solution is between 10⁶ and 10⁹ CFU/ml.

3. The process according to claim 1 or 2 wherein the probiotic lactobacilli are selected from the group consisting of Lactobacillus paracasel and Lactobacillus plantarum.

4. The process according to claim 3 wherein the probiotic lactobacilli are Lactobacillus paracasel LMGG P-22043 and Lactobacillus plantarum LMG P-22033.

5. The process according to claim 4 wherein the NaCl concentration in the brine solution is comprised between 4 and 5%.

6. The process according to claim 5 wherein the darkening inhibitor is citric acid.

7. The process according to claim 6 wherein the brine solution further contains spices and/or aromas.

8. The process according to claim 1 wherein the vegetable is selected from chicory, carrots, beetroot, potatoes, cauliflowers, wild onions, brussel sprouts, onions, tomatoes, peppers, aubergines, zucchini, cucumber, capers, mushrooms, asparaguses and artichokes.

9. The process according to claim 8 wherein the vegetable is artichoke.

10. Vegetable preserves obtained through the process of claim 1.

11. Vegetables treated with cultures of Lactobacillus paracasei and Lactobacillus plantarum strains.

12. Vegetables according to claim 11 wherein the strains are selected from the group consisting of Lactobacillus paracasei LMG P-22043 and Lactobacillus plantarum LMG P-22033.

13. Vegetables according to claim 10 or 11, wherein the vegetables are artichokes.

14. Lactobacillus plantarum deposited with the Belgian Coordinated Collections of Microorganisms, accession number LMG P-22033.
